(19) United States
(12) Patent Application Publication
(10) Pub. No.: US 2017/0296384 A1
(43) Pub. Date: Oct. 19, 2017

(54) TREATMENT SYSTEM FOR OPHTHALMIC PURPOSES WITH STORAGE

(71) Applicant: Carl Zeiss Meditec AG, Jena (DE)

(72) Inventors: Georg FLEISCHMANN, Essenbach (DE); Dietmar STEINMETZ, Bucha (DE)

(21) Appl. No.: 15/489,252

(22) Filed: Apr. 17, 2017

(30) Foreign Application Priority Data
Apr. 19, 2016 (DE) 10 2016 206 568.8

(51) Int. Cl. A61F 9/008; A61F 9/007; A61B 3/10

(52) U.S. Cl. CPC A61F 9/00821; A61F 9/00781; A61B 3/102; A61F 2009/00851

(57) ABSTRACT

A treatment system for ophthalmic purposes with a base body and an articulated arm with an applicator for applying an examination or treatment to a patient's eye, which includes a locking device for the secure storage of the articulated arm.

### CROSS-REFERENCE TO RELATED APPLICATION

The present application claims priority to German Application No. DE 10 2016 206568.8, filed Apr. 19, 2016, said application being incorporated herein in its entirety by reference.

### TECHNICAL FIELD

The invention relates to a treatment system for ophthalmic purposes with a base body and an articulated arm, wherein the articulated arm comprises an applicator for applying an examination or treatment to a patient's eye in a patient's head, and further a device for the secure storage of the articulated arm.

### BACKGROUND

For a number of years, devices and methods for the examination and treatment of eye diseases have been developing both towards a higher complexity and towards a more compact embodiment: The space taken up by a treatment system for ophthalmic purposes should be as small as possible, as such treatments frequently need to be carried out in a sterile environment. On the other hand, such systems should also be used such that the position of the patient must not be changed. Specifically for methods, in which examination and treatment are alternated, this is a major advantage as the used subsystems can then be calibrated with one another.

In ophthalmological laser treatment systems, it has now become standard to combine a laser treatment device to generate cuts in an eye tissue, or to ablate or coagulate an eye tissue using laser radiation with an examination device and to preferably integrate them into one common system.

For example, this is very advantageous in laser-assisted eye surgery to correct defective vision or to treat other eye diseases, such as a cataract using cataract surgery, where work steps to characterize the eye structures alternate with surgical steps and steps to verify the surgical intervention or support the surgical intervention. In this way, the eye structures can initially be characterized using optical coherence tomography (OCT) or using ultrasound. An eye tissue can subsequently be cut using a pulsed laser beam, in this case be separated for example using photodisruption. The result can be verified using a surgical microscope and subsequent steps, such as for example in cataract surgery the suction of an eye lens being previously cut with a laser beam and/or a cloudy eye lens being disintegrated by ultrasound, can be carried out under supervision by the surgical microscope.

To this end, a treatment system can be used comprising adjustable articulated arms, comprising in turn an appropriate applicator that is positioned on the patient's eye such that a treatment laser radiation for example is focused in an eye tissue or else the eye in question can be monitored using a surgical microscope. Specifically when using a treatment laser radiation for ophthalmic surgical purposes, it is advantageous to establish a fixed relative position between the eye to be treated and the applicator of the treatment laser beam. This is generally achieved by a fluid patient interface or in a simple way using a contact lens, with which the eye is fixed to the applicator of the articulated arm in question.

However, the applicator or articulated arm must also be securely fixed in the resting position. However, it should also be easily removable from its anchorage.

Should the articulated arms be coupled, as is practical for example in cataract surgery, where for example a surgical microscope being arranged on a second articulated arm can be connected with the laser applicator being arranged to a first articulated arm by a coupling device, an unbalanced weight compensation of the second articulated arm for example if this is manually adjustable can lead to a sudden transgression of a reliable compressive force on the patient's eye.

### SUMMARY

Embodiments of the present invention therefore describe a treatment system and a device for secure storage of the articulated arm, which avoids the aforementioned problems and requires no interaction with external equipment.

A treatment system for ophthalmic purposes comprises a base body and an articulated arm, wherein the articulated arm comprises a moveable or immovable end being fixed to the base body, a free end and articulated members being moveable relative to one another.

Treatments comprise examinations, specifically for the characterization of the structures and tissue of the eye, monitoring processes as well as treatments and specifically surgical application such as laser treatment, for example.

The articulated arm is moveable within the room. The treatment system comprising the articulated arm can also comprise additional articulated arms in addition to this articulated arm; it comprises therefore at least one articulated arm.

The articulated arm can be attached directly or indirectly to the base body with a fixed end: If it is indirectly attached to the base body, the base body comprises an extension for this purpose, for example in the form of a column or axle rod protruding from the base body. The articulated arm is indirectly attached to the base body using this extension.

An end of the articulated arm, being moveably fixed to the base body, thereby defines an anchorage, at which the articulated arm comprises an axle or Cardan joint directly at the point of anchorage, around which it can be swiveled or rotated. An end of the articulated arm, being immovably fixed to the base body, means a rigid attachment of this end, such that it can only change the location of the articulated arm in the room with its first articulated member.

Individual articulated members are thereby connected with one another by rotary ball swivels or rotary axles containing hinges.

The articulated arm of the treatment system further includes an applicator for applying an examination or treatment to a patient's eye in a patient's head and a sensor for measuring a force and/or relative movement. The applicator and sensor are for example arranged on the free end of the articulated arm.

At first, the term applicator specifies a device, from which an interaction of the treatment system with the patient's eye occurs. For example, the applicator denotes an exit location of an examination and/or treatment radiation or else the location from which an observation of the patient's eye is possible.

The free end of the articulated arm is the end opposite the end of the articulated arm that is moveably or immovably fixed to the base body of the treatment system. An arrangement on the free end of the articulated arm does not necessarily mean only an arrangement directly on the end of the articulated arm, but rather incorporates the surroundings of this free end: For example, the applicator can be located directly on the free end of the articulated arm, with the sensor on the other hand being at a somewhat engaged position.

In a preferred treatment system, the base body contains a treatment laser source to produce a treatment laser radiation. This is specifically a short-pulse laser radiation, for example a femtosecond or picosecond laser radiation. The base body and the articulated arm also therefore comprise a device for controlled focusing and deflection of the treatment laser radiation, wherein the applicator is arranged such that the treatment laser radiation exits from the treatment system through the applicator in the direction of the patient's eye; the focus of the treatment laser radiation can therefore be moved in the patient's eye.

Of particular advantage is a treatment system containing a first articulated arm with an applicator for applying a treatment laser radiation to a patient's eye and a second articulated arm with an examination device, such as a surgical microscope. An option to couple the applicator with a surgical microscope for applying a treatment laser radiation offers the advantage of examining the patient's eye using the second articulated arm with the surgical microscope, of working on said eye using the applicator for applying the treatment laser radiation, i.e. performing cuts in a tissue of the patient's eye for example, and of observing the effect of the treatment laser radiation in the patient's eye with the surgical microscope by coupling the two arms during the use of the applicator for applying the treatment laser radiation or between two operations of treatment laser radiation.

Instead of the surgical microscope or as well as the surgical microscope, other examination options, such as ultrasound or optical coherence tomography (OCT) can also be used accordingly.

If two articulated arms are coupled together, the treatment system is arranged such that the weight compensation and effect of the protective device can be applied to the coupled system of two articulated arms.

The locking device per the invention is characterized in that it comprises two preferably L-shaped levers, which are rotatable around an axis being located in proximity to the intersection of its brackets and comprise an axis in proximity to its upper end, around which ratchet levers are rotatable, which are spring-braced against each other, wherein the L-shaped levers can clamp a removable bar (290) with their other end and said device is arranged such that the removal of a fastening element being connected with the applicator is prevented in a first position by the ratchet levers, and the removal of the fastening element (320) is releasable in a second position after removing the bar.

Thus, the locking device is advantageously arranged such that the release to remove the fastening element is only possible if no force is being exerted on the ratchet levers by the fastening element.

In a further advantageous way, the locking device is arranged such that upon impact of a force from the fastening element to the ratchet levers via limit stops the L-shaped levers are distorted such that the bar is clamped and therefore no longer removable.

In another further advantageous way, the locking device is arranged such that the fastening element can be inserted in the first position.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention in question will now be explained on the basis of example embodiments. It can be seen that:

FIG. 1 depicts an example embodiment of a treatment system per the invention with a locking device;

FIG. 2 depicts an example embodiment of the locking device of a treatment system per the invention;

FIG. 3 depicts the locking device from FIG. 2 with the removal of the laser arm; and

FIG. 4 depicts the locking device from FIG. 2 with the storage of the laser arm.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

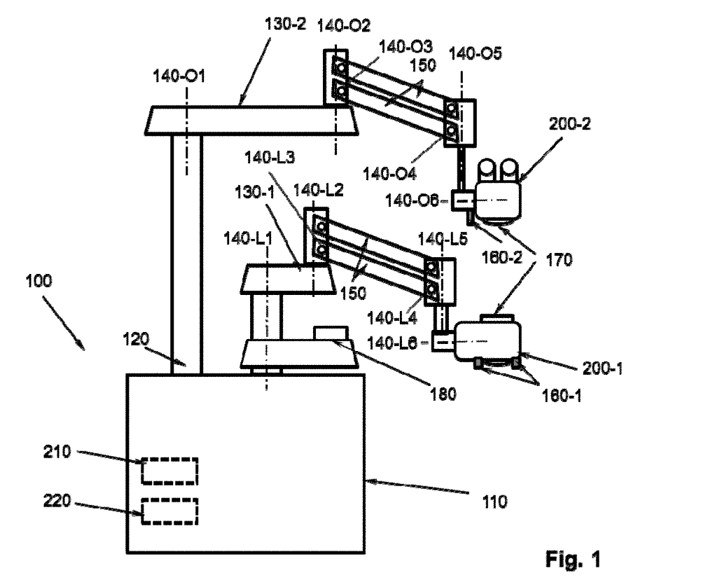

In FIG. 1, an example embodiment of a treatment system per the invention for ophthalmic purposes 100 is shown. In this example embodiment, the treatment system 100 comprises a base body 110 an articulated arm 130-1 with a laser applicator 200-1 to apply a treatment laser radiation to the patient's eye and a further articulated arm 130-2 with a surgical microscope 200-2 as the applicator of the further articulated arm 130-2. Both articulated arms 130-1, 130-2 are attached respectively with an end of the articulated arm 130-1, 130-2 by an extension 120 to the base body 110, while the laser applicator 200-1 and the surgical microscope 200-2 is arranged respectively to the other free end of the articulated arm 130-1, 130-2. The articulated arms 130-1, 130-2 comprise articulated members that are moveable relative to one another, which are moveable around horizontal hinge axles 140-O3, 140-O4, 140-L3, 140-L4 and vertical hinge axles 140-O1, 140-O2, 140-O5, 140-L1, 140-L2, 140-L5.

The articulated arm 130-1, comprising the laser applicator 200-1, contains structures that deflect and focus the treatment laser radiation, which is produced in a femtosecond laser source 220 being arranged in the base body 110 of the treatment system 100. The laser applicator 200-1 contains the exit port of this controlled and focused femtosecond laser radiation. Through the relevant positioning over the patient's eye to be treated, such that the patient's eye is in a fixed relationship to the laser exit port, which occurs here by fixing the patient's eye to the laser applicator 200-1 by a patient interface (not shown here), attention is paid to ensuring that the focus of the femtosecond laser radiation can be moved in a defined pattern, wherein the eye tissue is separated by photodisruption at the relevant effective area of the focus of the femtosecond laser radiation. A cut in the tissue of the patient's eye thus occurs along the scan pattern of the femtosecond laser radiation. The generation of the femtosecond laser radiation and its deflection and focusing are controlled by a control unit 210, which also controls all other functions of the treatment system per the invention 100.

The herein-described example embodiment of the treatment system 100 contains furthermore a device for coupling the articulated arms 170 comprising two subelements, from which one is arranged on the upper side of the laser applicator 200-1 and the other is arranged on the lower side of the surgical microscope 200-2 such that the two subelements can interlock according to the lock-and-key principle. The surgical microscope 200-2 can thereby be coupled with the laser applicator 200-1 and the progression of the laser treatment, i.e. for example the progression of a cut in the cornea, sclera, capsular sac or eye lens, can be observed by the surgical microscope 200-2 by femtosecond laser radiation being applied with the laser applicator 200-1.

A force sensor device 160-1 is arranged on the underside of the laser applicator 200-1 of the articulated arm 130-1, i.e. on the eye side.

Both articulated arms 130-1, 130-2 include a weight compensation device 150, respectively. This is designed respectively in the form of a parallel support arm 150, which contains, for example, a compensation spring (not shown here). The parallel support arm 150 of the articulated arm 130-1 which comprises the laser applicator 200-1 substantially comprises four hinges: namely the first rotary head 140-L2 with the two parallel axles 140-L3, and the second rotary head 140-L5 with the two parallel axles 140-L4, and the articulated members, i.e. the spring arm 150-2 and the strut 150-1. The weight compensation for all masses to be compensated occurs relative to the hinge 140-L3.

The weight compensation of the surgical microscope articulated arm 130-2 can be manually adjusted and is adjusted by the operator to the relevant weight of the accessory being attached to the surgical microscope 200-2 or to the coupled articulated arm with laser applicator 200-1. Deviations from the optimal adjustment lead to residual upward- or downward forces, which can also be greater than the braking force of the surgical microscope. Residual downward forces are critical. The surgical microscope 200-2 with coupled applicator must not drop alone if it is released by the operator, specifically not at the moment of positioning over the patient's eye.

To rule out basic problems due to an inadequate weight compensation, a further force sensor is installed (not shown in FIG. 1) in a locking device, subsequently referred to as a parking basin 180, on which the articulated arm 130-1 with the laser applicator 200-1 can be stored in a resting position. This measures the residual forces of the surgical microscope 200-2 and coupled laser applicator 200-1 at the moment of coupling. To this end, the brakes of the articulated arm 130-2 containing the surgical microscope 200-2 are momentarily detached and the residual forces are calculated. If the deviations are greater than permitted, then a locking device of the laser applicator 200-1 in the parking basin 180 is not opened. The parking basin 180 recognizes whether the laser applicator 200-1 is present or not on the basis of the installed inductive force sensor 230. When the residual forces are within the permitted range, the locking device of the parking basin 180 is only momentarily opened long enough for the laser applicator 200-1 to be removed. Thereafter, the locking mechanism is reclosed. The laser applicator 200-1 on the articulated arm 130-1 can then be stored, but not removed again. Thus, removal is only possible in a controlled, balanced state.

Should the system, e.g. due to malfunction, become electrically disconnected when the laser applicator 200-1 is still in the rest position in its parking basin 180, then the locking device mechanism in the parking basin 180 retains the laser applicator 200-1 and its articulated arm 130-1. This cannot be outwardly perceived.

Only when the forces being measured by the force sensor in the parking basin 180 are within the required limit is the locking device suspended for the removal of the laser applicator 200-1.

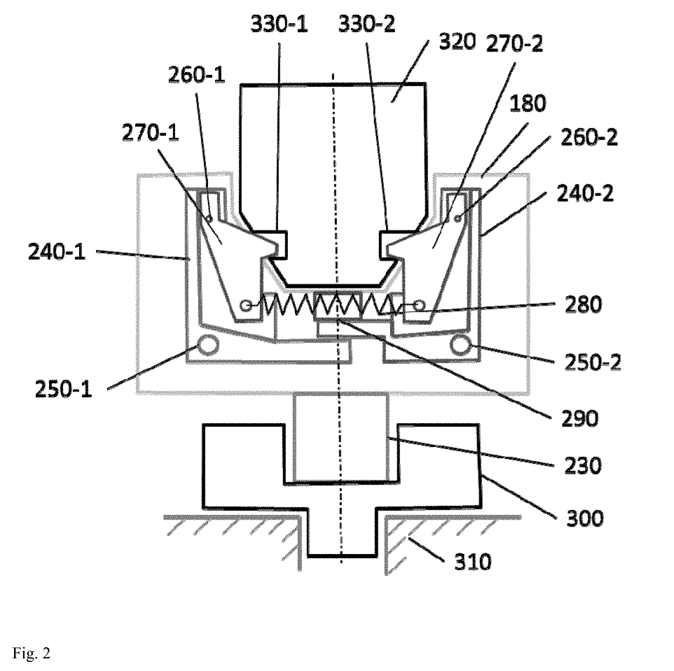

In FIG. 2, the locking device per the invention (parking basin) 180 is shown in more detail. In its interior the parking basin comprises two substantially L-shaped levers 240-1, 240-2, which are respectively rotatable around an axis 250-1, 250-2 being found in proximity to the intersection of the brackets of the lever 240-1, 240-2. In proximity to the ends of the vertical brackets, ratchet levers 270-1, 270-2 are rotatably stored on axles 260-1, 260-2, which are braced with one another through a tension spring 280. The tension spring 280 thereby presses the two levers 240-1, 240-2 against a bar 290 and said bar to the housing of the parking basin 230, wherein lever 240-2 supports lever 240-1. The bar 290 is removable using an actuating device not shown here, e.g. an electrically operated magnet. Using the force sensor 230, the parking basin 180 is connected with the housing 310 of the treatment device by a rotatable bracket 300. On the laser arm or applicator 200-1, a fastening element 320 is attached comprising grooves 330-1, 330-2, into which the locking elements of the ratchet levers 270-1, 270-2 engage by applying tension to the tension spring 280. In this state, the laser arm is locked and also cannot be removed by pulling upward (e.g. by maloperation or incorrectly adjusted spring compensation), as with the exertion of force of the grooves 330-1, 330-2 on the ratchet levers 270-1, 270-2 these rotate the L-shaped levers 240-1, 240-2 outward using limit stops 340-1, 340-2, which are closer to the end of the levers 270-1, 270-2 than the axles 260-1, 260-2, whereby the bar 290 is clamped such that the pull magnet also cannot be removed from it.

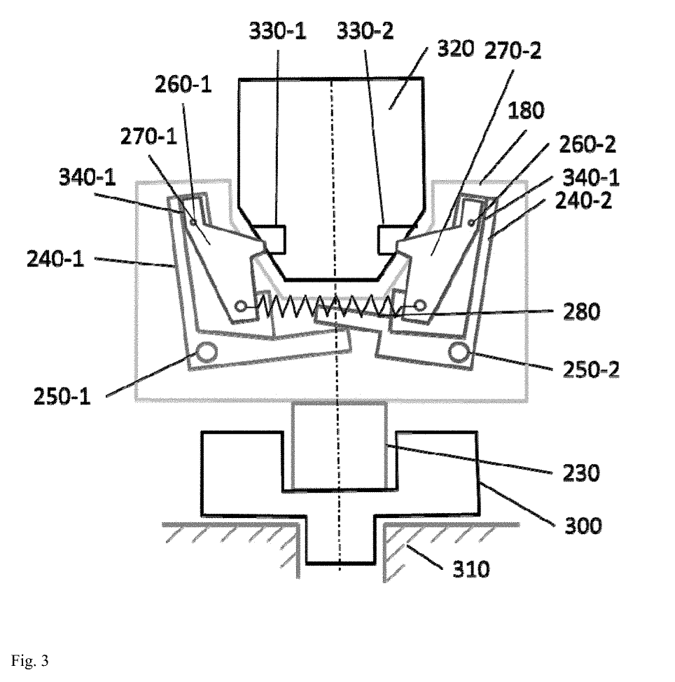

Only when the weight compensation is produced and therefore no force exerted from the fastening element 320 onto the ratchet levers 270-1, 270-2 can the bar 290 be removed by the magnet and the laser arm can be extracted. This situation is shown in FIG. 3. By pulling the laser arm with the fastening element 320 upwards, the two ratchet levers 270 are pushed apart from the tension spring 280 such that the laser arm can be removed. Then the bar 290 can be reinserted, as the tension spring 280 pulls the ratchet levers 270-1, 270-2 back together and thereby the L-shaped levers 240-1, 240-2 are also returned to the starting position, whereby the parking basin 180 is again ready to store the laser arm.

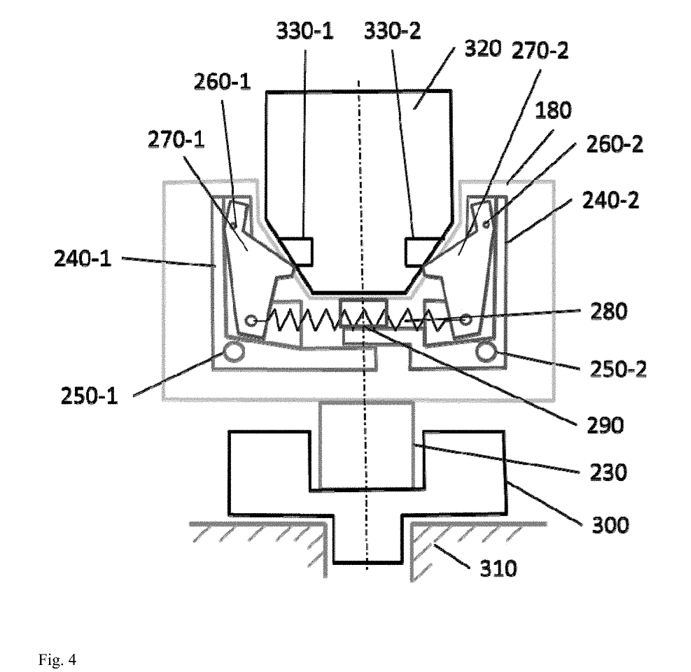

As is shown in FIG. 4, when storing the laser arm, the ratchet levers 270-1, 270-2 are pressed outwardly until the locking elements are contracted by the spring 280 and engage in the grooves 330-1, 330-2 of the fastening element 320 of the laser arm, thereby locking this again. Thereafter, the situation shown in FIG. 2 is attained again.

Furthermore, the parking basin 180 may comprise another sensor, which detects the presence of the laser arm. This can be inductive or capacitive or also a microbutton. The two L-shaped levers 240-1, 240-2 do not have to brace against each other; they can also both brace against the bar 290. The magnet only requires brief impulses to pull the bar 290 from the engagement; in a currentless state a spring also not shown here ensures that it is returned to the starting position according to FIG. 2. Thereby, no unnecessary heat development and no power consumption occur and the pull magnet can be dimensioned in an appropriately space-saving fashion.

Another advantage of the suggested solution is that just one spring 280 suffices to pull all levers into the starting position.

The aforementioned features of the invention being explained in various example embodiments are thereby not only usable in the example stated combinations, but also usable in other combinations or alone, without departing from the scope of the present invention.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. §112(f) are not to be invoked unless the specific terms “means for” or “step for” are recited in a claim.

1.-10. (canceled)

11. A treatment system for ophthalmic purposes, comprising:
a base body and an articulated arm;
wherein the articulated arm includes a movable or immovable end that is fixed to the base body;
a free end and articulated members being moveable relative to one another;
an applicator that applies an examination or treatment to a patient's eye in a patient's head; and
a locking device, in which the applicator is storable.

12. The treatment system according to claim 11, wherein the locking device is structured to only release the applicator in a force-free state.

13. The treatment system according to claim 11, wherein the base body contains a treatment laser source to produce a treatment laser radiation; and
the base body and the articulated arm comprise a device for controlled focusing and deflection of the treatment laser radiation;
wherein the applicator is arranged such that the treatment laser radiation exits the treatment system through the applicator in a direction of the patient's eye.

14. The treatment system according to claim 11, wherein the laser source comprises a short pulse laser.

15. The treatment system according to claim 11, wherein the laser source comprises a femtosecond laser or a picosecond laser.

16. The treatment system according to claim 12, wherein the locking device further comprises a force sensor.

17. The treatment system according to claim 11, wherein the articulated arm further comprises a weight compensation device.

18. The treatment system according to claim 11, wherein the articulated arm comprises a device for coupling the articulated arm with a further articulated arm.

19. A locking device for a treatment system for ophthalmic purposes, comprising:
two levers, which are each rotatable around an axis and are located in proximity to an intersection of brackets and which comprise an axis in proximity to an upper end thereof, around which ratchet levers are rotatable, which are spring-braced against each other;
wherein the ratchet levers are structured to clamp a removable bar with another end of the ratchet levers and wherein said locking device is structured such that the removal of a fastening element is prevented in a first position by the ratchet levers, and the removal of the fastening element is releasable in a second position after removing the bar.

20. The locking device according to claim 19, wherein the levers are L-shaped.

21. The locking device according to claim 20, which is structured such that release to remove the fastening element is only then possible if no force is being exerted on the ratchet levers by the fastening element.

22. The locking device according to claim 21, which is arranged such that upon impact of a force from the fastening element to the ratchet levers using limit stops the L-shaped levers are distorted such that the bar is clamped.

23. The locking device according to claim 19, which is arranged such that the fastening element is insertable in the first position.
